(19) United States
(12) Patent Application Publication
(10) Pub. No.: US 2012/0209222 A1
(43) Pub. Date: Aug. 16, 2012

(54) DISPENSATION OF A PHARMACEUTICAL LIQUID

(76) Inventor: Giovanni Benedetti, West Kilbride (GB)

(21) Appl. No.: 13/370,392

(22) Filed: Feb. 10, 2012

(30) Foreign Application Priority Data
Feb. 11, 2011 (GB) 1102442.9

(51) Int. Cl. A61F 9/00; A61M 35/00

(52) U.S. Cl. 604/301; 604/295

(57) ABSTRACT

An assembly (1) for displaying and dispensing a pharmaceutical liquid contained in a single-use container (3), wherein said assembly (1) comprises: a dispenser comprising a frame (30) to hold said container (3) in a display position; and at least a single-use container (3) comprising a vessel (2) and a detachable closure element (6) which, once detached, defines an outlet to said vessel (2). The assembly (1) further comprises coupling means provided onto the container (3) and the detachable closure element (6) to mount the detachable closure element (6) onto the dispenser. The dispenser further comprises serving means to detach the closure element (6) from said vessel (3). These serving means are actuated by a lever and comprise a handle (33). The invention further relates to the said individual single-use containers as well as dispensers as described above.

### CROSS REFERENCE TO RELATED APPLICATIONS

The present application claims the benefit of United Kingdom Application Serial No. 1102442.9, filed Feb. 11, 2011, the disclosure of which is hereby incorporated by reference.

### RELATED ART

1. Field of the Invention

The present invention relates to a device for the dispensation of pharmaceutical liquids from single use containers.

2. Brief Discussion of Related Art

Pharmaceutical and/or first aid products can either be solid items such as bandages, wound dressings, pills and the like or liquid items such as eye rinsing liquid, saline solutions or drugs in a liquid form. In the latter case, liquid pharmaceutical items are often packaged in containers, such as bottles, flask or vials, which are designed to be used only once then disposed of. Single-use packaging can be used for various reasons such as to provide set doses, to maintain or improve sterility or to prevent tampering and/or degradation of the packaged liquid.

The provision of eyewash liquids or rinsing fluids is frequently required by health and safety legislation in some workplaces. Such liquids can be sterilized water or saline solutions and are provided to remove, dilute or neutralize harmful contaminants from the eye of a user. They are usually provided in an integral plastic container having the general shape of a bottle or a tube.

For example, U.S. 2005/0261640 describes a typical eyewash flask provided with a closure element which can be broken off from the flask by rotating it. In this document the closure element is torn away from the bottle via rotation of a sleeve which is secured to the closure element. Once torn, the closure element cannot be used to close the flask again.

Eyewash bottles are frequently displayed in a holder, which can be wall mounted, and which is positioned so that it allows easy access for the user in an emergency. Some of such devices further provide for the opening of the bottle to occur when it is taken away from the holder to prevent re-use of a fluid with compromised sterility. Such a device is described in WO2005/025480. In this document the closure element is either a liner or a tab which is engaged by the eyewash bottle holder and becomes separated when the user pulls the bottle away from the holder.

A problem associated with the dispensation of pharmaceutical liquids is that known devices usually require the use of two hands, one to hold the container and the other to open it. As the user, especially in case of an emergency, may not have the use of both his/her hands, these devices are unsatisfactory.

Therefore eyewash containers provided with detachable closure elements are twistable and the closure element is torn off the bottle by rotation/twisting. These types of bottles are unsatisfactory because it is difficult to achieve the grabbing and twisting motion when the bottle is provided in a wall-mounted holder. In this case both hands may be needed. Also it requires the user to have the presence of mind and/or prior knowledge that a twisting motion, and not a straightforward pulling motion, is required. In an emergency this may not be the case, especially if the user is blinded to some degree and cannot see the instructions provided. Moreover, the closure element is usually firmly attached to the bottle to avoid that this closure element becomes accidentally detached and consequentially force is usually required by the user.

Furthermore, when holders are used to hold and display the containers, holding and supporting features may obstruct direct access to the container by the user. This, in the case of an emergency, is highly undesirable.

Therefore there is still a need for a single-use container and dispensing device of single-use containers which suppress or alleviate at least some of the drawbacks of known devices and which provide a direct, speedy, simple and hygienic access to a pharmaceutical or first aid fluid whilst being both reliable and inexpensive.

### INTRODUCTION TO THE INVENTION

According to a first aspect of the invention it is provided an assembly for displaying and dispensing a pharmaceutical liquid contained in a single-use container, wherein said assembly comprises: (a) a dispenser comprising a frame to hold said container in a display position; and, (b) at least a single use container comprising a vessel and a detachable closure element which, once detached, defines an outlet to said vessel, said assembly further comprising coupling means provided onto said container and said detachable closure element to mount said detachable closure element onto said dispenser, said dispenser further comprising severing means to detach the closure element from said vessel which are actuated by a lever, which comprises a handle.

The provision of a lever onto the dispenser to actuate the severing means is particularly advantageous in case of an emergency as it can be prominently positioned and allow a single, easy, simple and rapid motion to sever the closure means and access the pharmaceutical liquid. The handle provided onto the lever preferably comprises a rotating handle.

Another object of the invention is a single-use container which can be used in an assembly of the invention.

A further object of the invention is a dispenser which can be used in an assembly of the invention.

The container of the invention is configured for single use and can be tamper evident or re-seal resistant. Preferably, the detachable closure element is integrally molded with the vessel and may be made of one integral sheet of plastic materials. Advantageously a weakness line (e.g. recess, groove or score line) is provided between the detachable closure element and the vessel to case the separation between these two parts. It is preferred that a grippable part, such as a tab (or flange) be provided to the detachable closure element.

The pharmaceutical liquid being held by the container is preferably a suitable eyewash fluid such as sterilized water or a saline solution.

It is further preferred that the coupling means provided onto said detachable closure element are configured to mount the container onto said dispenser and comprise a cap or a clip which can be secured (e.g. snap fitted) to the detachable closure element and advantageously to its grippable part when provided with one. The coupling means can comprise a clipping arrangement whereas said dispenser is configured to receive and engage said cap or clip. Thus, for example, at least one tongue may extend upwardly from said detachable closure element and be received by a cooperating flange or rail provided onto said dispenser in a snap fit or a sliding relationship.

Advantageously the severing means provided onto the dispenser of the invention cooperates with the coupling means.

According to a further embodiment of the invention, the severing means further comprise means to hold the vessel in position with respect to said severing means when they are actuated.

According to a further embodiment of the invention the dispenser comprises a cradle to receive and hold the container, once opened, in a dispensing position.

According to yet a further embodiment of the invention, the dispenser comprises a protective cover which is configured to inhibit direct access by a user to, at least, the detachable closure element of said container when said container is held by the dispenser in its display position. The protective cover can also protect the detachable closure element and its surroundings from contamination such as dust and dirt.

According to a further embodiment of the invention the dispenser may comprise a release mechanism allowing the release of the container from its display position without actuation of the severing means. Such a release mechanism can comprise a pressure-actuated release button connected to a release member which can disengage the coupling means.

According to a first particularly preferred embodiment of the invention, the vessel is preferably in the shape of a bottle, or a flask, provided with a vertical axis and may hold more than 200 ml of liquid (preferably 500 ml or more). The detachable closure element is usually provided at the top of the bottle. Advantageously the frame comprises holding means sized and shaped to prevent the rotation of the bottle around its vertical axis. According to this first embodiment, the holding means of the frame comprise a cradle which is sized and shaped to engage the bottom part of the bottle and which may be slidably mounted onto the dispenser so as to be able to move from the display position to a dispensing position.

According to this first particularly preferred embodiment of the invention the handle and the severing means are rotationally mounted onto the frame of the dispenser along an axis substantially parallel, and preferably co-axial, to the one of the bottle. It is further preferred that said severing means are mounted onto said detachable closure element so that, in operation, rotation of the handle exerts a rotational force or twisting action upon said element.

According to this first particularly preferred embodiment, the container may comprise an eye cup which can be positioned around the outlet of the container and/or around said detachable closure element.

According to a second particularly preferred embodiment of the invention the vessel is preferably tubular in shape and can hold no more than 50 ml of liquid (preferably about 20 ml). Preferably the detachable closure element is provided at one of the extremities of the tube.

According to this second particularly preferred embodiment, the coupling means comprise a rail mounted onto said dispenser and a groove or hook provided on the detachable closure element and which is configured to engage said rail. It is further preferred that the assembly comprises a series of containers which are engaged by and aligned along said rail.

According to this second particularly preferred embodiment, the severing means include holding means which comprise a blocking surface, or a casing, which engages the vessel and restrains or prevents its movements relative to the severing means. The severing means further comprise a cutting member which, upon actuation by a rotating handle, will pivot, abut and force said closure element away from the vessel thereby detaching it therefrom. Advantageously a tab portion is provided onto the detachable element and comprises a contact surface configured to engage with said cutting member.

According to this second particularly preferred embodiment, at least one weakness line can be provided between the detachable closure element and the vessel and the cutting member comprises a folding member which exerts, upon actuation of the lever, a force which is normal to said weakness line resulting in the folding, and at least partial tearing, of the detachable closure element along said weakness line. It is preferred that the surface engaged by the folding means be provided away from the weakness line thus allowing for the tab to pivot with respect to the vessel and for a further lever action to take place.

Additionally a severing element can be provided to complement the action of the folding element. This severing element is positioned staggered from the folding element and is sized arid shaped to abut, upon rotation of the handle, but after the folding has taken place, the detachable closure element along the weakness line and complete the severance of the folded detachable closure element and the opening of the container.

According to a preferred embodiment of the invention, said severing means are pivotally mounted onto said frame and their axis of rotation is co-axial with the axis of rotation of the rotating handle.

### BRIEF DESCRIPTION OF THE DRAWINGS

FIG. 1 shows a perspective view of an assembly according to an embodiment of the invention.

FIG. 2 shows a perspective view of the single-use container which is part of the assembly of FIG. 1.

FIG. 3 shows an exploded view of the container of FIG. 2.

FIG. 4 shows a fragmentary perspective and cross sectional view of the top part of the container of FIG. 2 taken along the line A-A′.

FIG. 5 shows a fragmentary front and cross sectional view of the top part of the container of FIG. 2 taken along the lines A-A′.

FIG. 6 shows a variant of the single-use eyewash container shown in FIGS. 2 to 5.

FIG. 7 shows a perspective view of the assembly of FIG. 1, the assembly being in loading position.

FIG. 8 shows a fragmentary cross sectional and perspective View of the top part of the assembly of FIG. 7 taken along the lines B-B′ of FIG. 1.

FIG. 9 shows the assembly of FIG. 7 to 10, where the severing mechanism has been actioned and the container is opened and ready for use.

FIG. 10 shows a fragmentary back cross sectional view of the top part of the assembly of FIG. 1 taken along the lines C-C′.

FIG. 11 shows a view similar to the one shown in FIG. 10, the release button being actuated.

### DETAILED DESCRIPTION

The exemplary embodiments of the present disclosure are described and illustrated below to encompass a device for the dispensation of pharmaceutical liquids from single use containers. Of course, it will be apparent to those of ordinary skill in the art that the embodiments discussed below are exemplary in nature and may be reconfigured without departing from the scope and spirit of the present disclosure. However, for clarity and precision, the exemplary embodiments as discussed below may include optional steps, methods, and features that one of ordinary skill should recognize as not being a requisite to fall within the scope of the present disclosure.

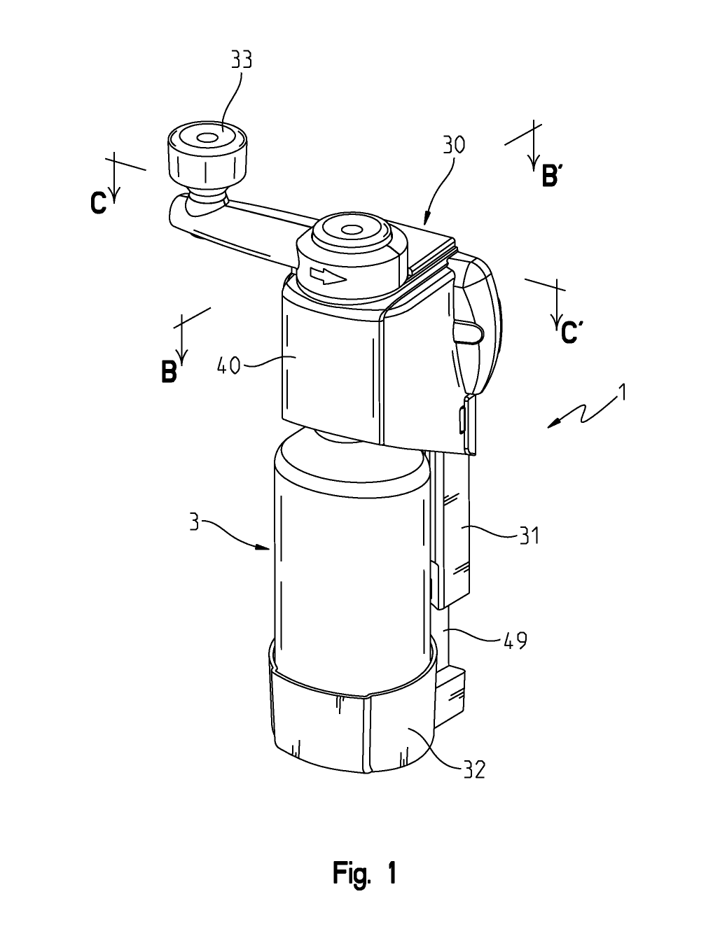

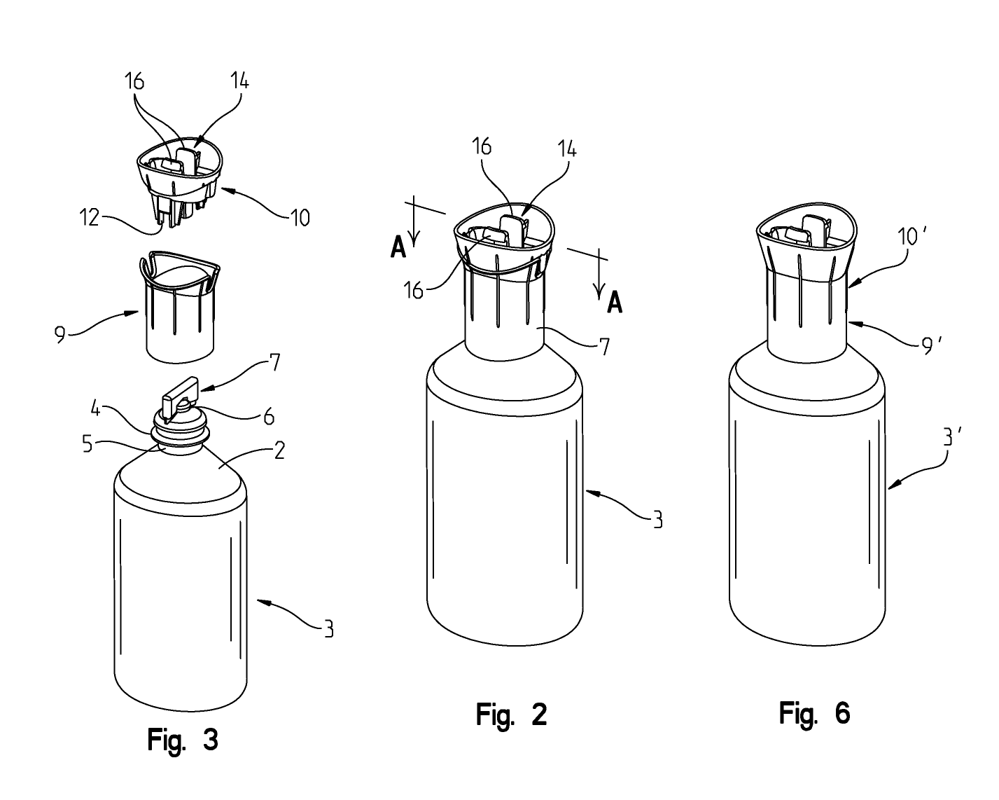

According to a preferred embodiment shown in FIGS. 1 to 11, the assembly 1 comprises a dispenser having a base or frame 30 which holds a container 3 containing an eyewash fluid such as sterilized water. As it can be better seen in FIG. 3, the container 3 adopts the overall shape of a bottle having a body or vessel 2 which comprises a neck 5 and is preferably not circular in cross section. As can be best seen in FIGS. 2, 3 and 6, the vessel 2 may have an elliptical or ovoid-shaped body. The detachable closure element 6 is positioned at the extremity of neck 5. A tab 7 is provided onto the detachable closure element 6. The closure element 6 is designed so it can be torn off from the remaining part of container 3, for example by providing weakness lines where the closure element 6 is attached to the remaining part of container 3 (i.e. vessel 2).

A cap 10 is firmly and permanently attached to the closure element 6, via a snap fit, thus forming one integral piece with the closure element 6. Cap 10 engages tab 7 via groove 12. Attaching means 14 are positioned to secure the detachable closure element 6 to the frame 30 and are advantageously positioned onto cap 10. As shown in FIGS. 2 to 5, attaching means 14 are conveniently in the shape of a pair of tongues/clips 16 which extend upwardly from cap 10.

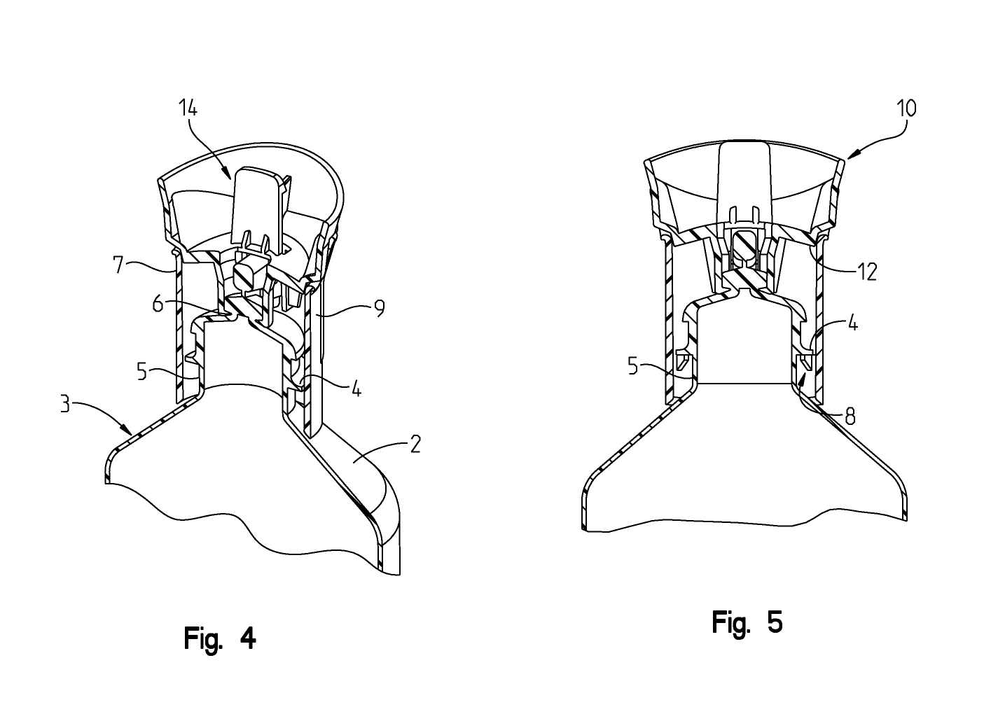

As best shown in FIGS. 4 and 5, a sleeve 9 can be permanently secured onto the vessel 2 and around the neck 5 in a snap fit arrangement between flange 8 and collar 4, in order to form an integral eye cup. Provision of an eye cup is however only a variant of the invention and can be omitted altogether as shown in FIG. 6. In this variant, sleeve 9′ is integral to cap 10′ and is not directly connected to container 3′.

Although cap 10 is slidably engaged by sleeve 9, there is no other connection between cap 10 and vessel 2 than via the detachable closure element 6.

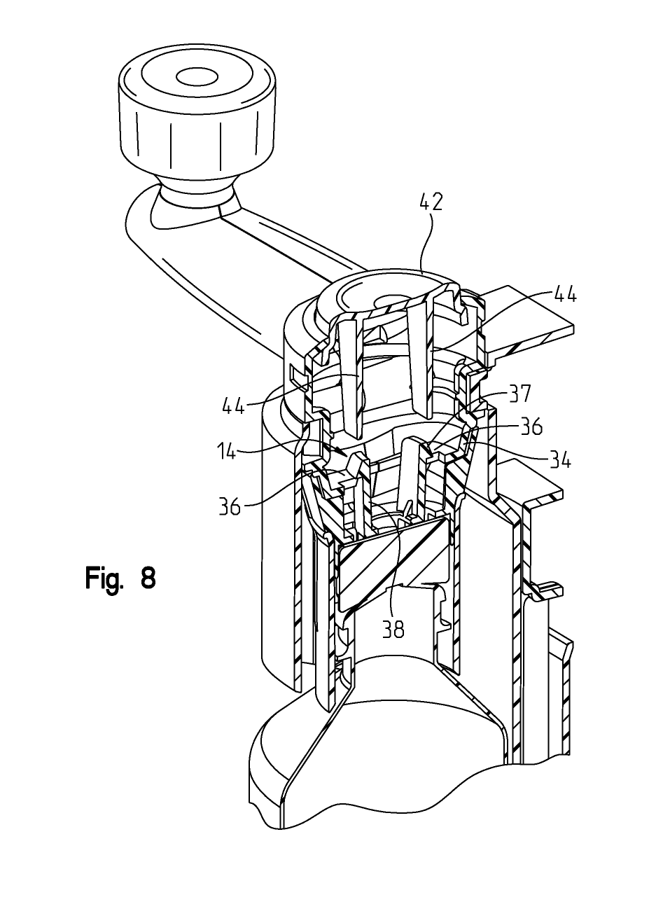

As best seen in FIG. 1 or 8, frame 30 comprises a main body 31 which is adapted to be wall mounted, although this is not a necessary requirement. Coupling means 34 extend outwardly from the main body 31. These coupling means 34 comprise an aperture 38 and flanges 36 which are configured to receive cap 10. Container 3 can be secured onto frame 30 by engaging the attaching means 14 onto the corresponding coupling means 34. More specifically, tongues 16 are provided through aperture 38 and engage retaining flanges 36. Tongues 16 are provided with some resiliency which allows the tongues 16 to clip into place and engage the retaining flanges 36.

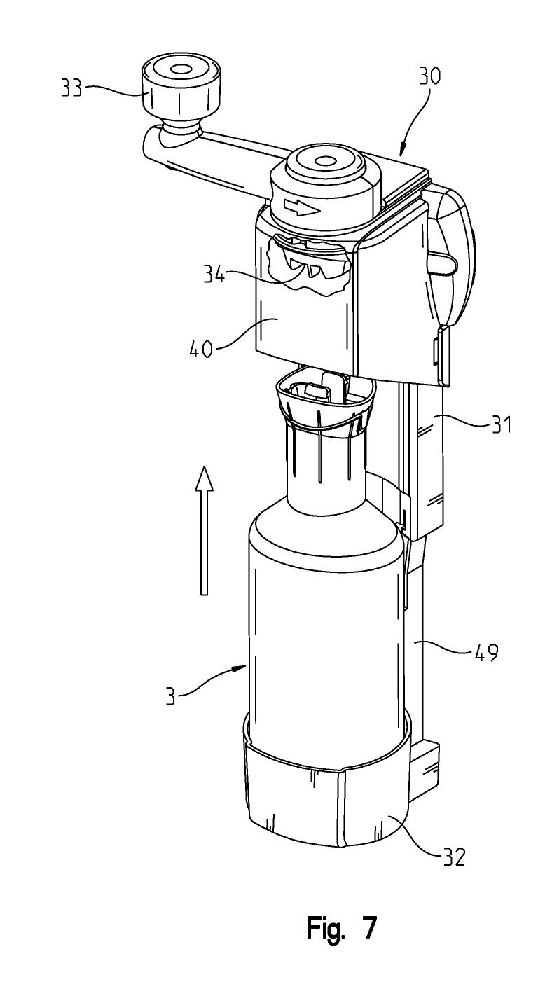

As best seen in FIGS. 1 and 7, a protective hood 40 is provided to protect the top portion of container 3. Protective hood 40 is mounted onto frame 30 and achieves the dual purpose of protecting the top of the container 3, the surrounding of the outlet and the eyecup from dust and infection, as well as preventing a user attempting to remove container 3 by pulling it directly from the base 30 without using the severing means (i.e. the handle 33).

Container 3 is also secured to the frame 30 via holding means, such as cradle 32, which receive and hold the bottom of container 3. According to a particularly preferred embodiment of the invention, the cradle 32 is vertically and slidably mounted onto the frame 30 via sliding rail 49. Hence, it can move away from the coupling means 34 and adopt a loading position where container 3 is positioned within cradle 32 away from coupling means 34. Once container 3 is loaded, the cradle 32 is moved upwardly by sliding cradle 32 along the rails 49 so that the attaching means of container 3 engage the coupling means 34, thereby securing container 3 onto the base 30 into a stowed or display position.

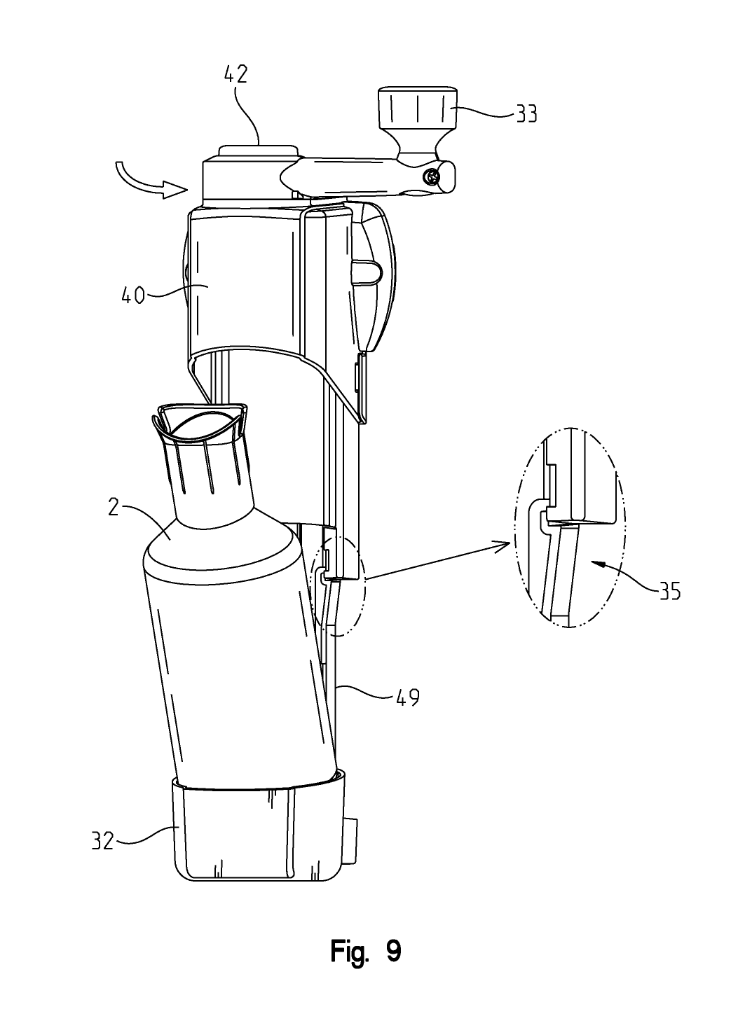

A handle 33 extends outwardly from base 3. The handle 33 is positioned, sized and shaped so as to be of easy reach and manipulation for a potential user. The handle 33 is connected to the coupling means 34 and both can rotate with respect to the frame 30 and cradle 32. In use, the container 3 is connected to the frame 30 through coupling means 34. In operation, if eyewash liquid is required in an emergency, the closure element 6 can be detached easily and quickly by rotating the handle 33. Rotation of the handle actuates rotation of coupling means 34, which in turn applies a rotational movement to cap 10. As containers is firmly maintained in position with respect to frame 30 by the cradle 32, the handle exerts a twisting motion upon closure element 6 via cap 10 and tab 7. As handle 33 pivots around container 3 (as. shown in FIG. 9) the closure element 6 is torn away from vessel 2 which is opened and released. The cap 10, together with tab 7 and severed closure element 6, remain coupled to the coupling means 34 within the dispenser. Due to gravity, the opened vessel 2 and the cradle 32 slides along rail 31 to adopt the dispensing position shown in FIG. 9, and is ready to use. The tilting of the bottle is carried out by the user by hand.

A latching mechanism 35 can advantageously be provided onto base 30 to lock the cradle 32 in a dispensing position and ease the removal of container 3 thereof by maintaining cradle 32 in position arid preventing it from sliding back up.

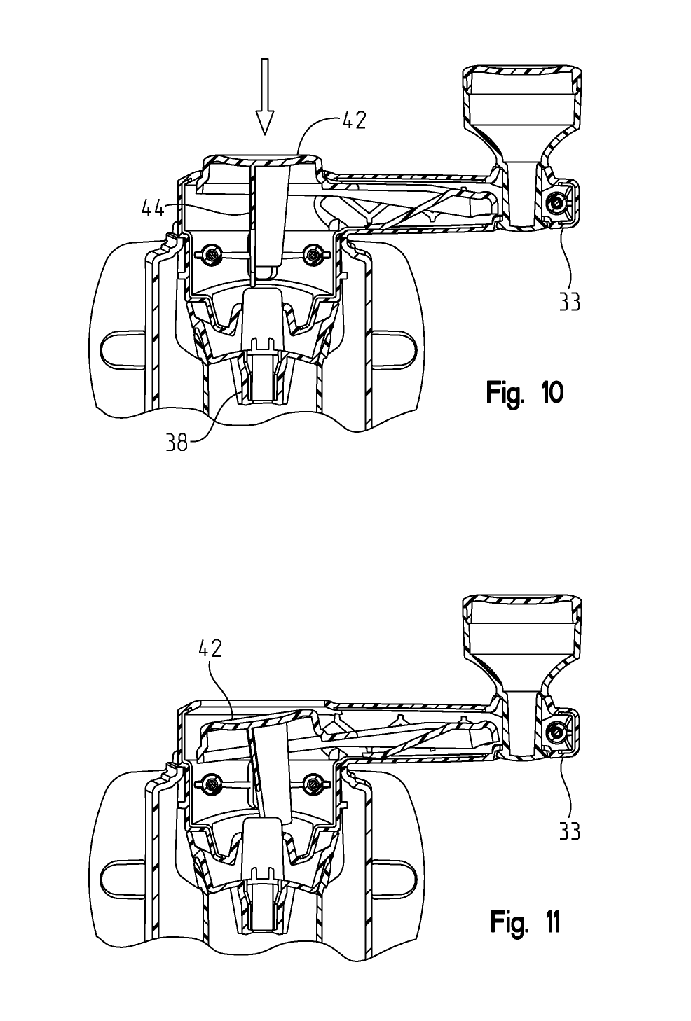

As best shown in FIG. 8 a release button 42 is conveniently provided at the top of the base 30 and opposite the cap 10. The release button 42 can be used to release the cap 10 when the container 3 has been used in order to load a new eyewash container. It can also be used to release the entire container 3 without opening it, for example if the container has passed its use-by-date and needs to be replaced. Release members 44, best shown in FIG. 8, project downwardly from, and are actuated by, said release button 42. In operation, when button 42 is depressed (see FIG. 11) by a user, the release members 44 are pushed downward and act upon tongues 14 to release them from coupling means 34. As shown in FIGS. 10 and 11, the release members 44 exerts a lateral force upon said attaching means 14 and disengage (un-clip) them from retaining flange 36. Once the attaching means 14 are disengaged, the cap 10 and the unopened container 3 is freed from the base 30. As shown in FIGS. 10 and 11, the release mechanism is conveniently provided at the top of handle 33 and the body 31.

The various elements that constitute the assembly of the invention can be made from a variety of materials such as plastic, metal, composite, glass etc. Preferably the container is made from blown molded propylene and the remaining part from high density polyethylene which are shaped by injection molding. Other well known materials can be used including various thermoplastic resins, homo and co-polymers etc.

Many modifications and variations of the present invention are possible in light of the above teachings. For example the particular shape of the attaching and coupling means can vary. It is therefore to be understood within the scope of the attached claims that the invention may be protected otherwise than specifically described in the above embodiments.

Following from the above description and invention summaries, it should be apparent to those of ordinary skill in the art that, while the methods and apparatuses herein described constitute exemplary embodiments of the present invention, the invention is not limited to the foregoing and changes may be made to such embodiments without departing from the scope of the invention as defined by the claims. Additionally, it is to be understood that the invention is defined by the claims and it is not intended that any limitations or elements describing the exemplary embodiments set forth herein are to be incorporated into the interpretation of any claim element unless such limitation or element is explicitly stated. Likewise, it is to be understood that it is not necessary to meet any or all of the identified advantages or objects of the invention disclosed herein in order to fall within the scope of any claims, since the invention is defined by the claims and since inherent and/or unforeseen advantages of the present invention may exist even though they may not have been explicitly discussed herein.

What is claimed is:

1. An assembly for displaying and dispensing a pharmaceutical liquid contained in a single-use container, wherein said assembly comprises:
a dispenser comprising a frame to hold said container in a display position; and,
at least a single-use container comprising a vessel and a detachable closure element which, once detached, defines an outlet to said vessel;
said assembly further comprising coupling means provided onto said container and said detachable closure element to mount said detachable closure element onto said dispenser;
said dispenser further comprising severing means to detach the closure element from said vessel which are actuated by a lever and which comprises a handle.

2. The assembly of claim 1, wherein said handle is a rotating handle.

3. The assembly of claim 1, wherein a weakness line is provided between the detachable closure element and the vessel.

4. The assembly of claim 1, wherein a tab is provided to the detachable closure element.

5. The assembly of claim 4, wherein said coupling means provided onto said detachable closure element comprise a cap or a clip which is secured to said tab.

6. The assembly of claim 5, wherein said coupling means comprise a clipping arrangement wherein said dispenser is configured to receive and engage said cap or clip.

7. The assembly of claim 1, wherein said severing means provided onto the dispenser of the invention cooperate with said coupling means.

8. The assembly of claim 1, wherein said severing means further comprise means to hold the vessel in position with respect to said severing means.

9. The assembly of claim 1, wherein said dispenser comprises a cradle to receive and hold the container, once opened, in a dispensing position.

10. The assembly according to claim 1, wherein the dispenser comprises a release mechanism which comprises a pressure-actuated release button connected to a release member which can disengage said coupling means.

11. The assembly according to claim 9, wherein said cradle is slidably mounted onto the dispenser so as to he able to move from said display position to a dispensing position.

12. The assembly according to claim 1, wherein said handle and severing means are rotationally mounted onto the frame of the dispenser along an axis substantially parallel, and preferably co-axial, to the one of the container.

13. The assembly according to claim 1, wherein said vessel comprises an eye cup which is mounted around the outlet of the container and/or around said detachable closure element.

14 A single-use container as described in claim 1.

15. A dispenser as described in claim 1.
